(19) United States
(12) Patent Application Publication
(10) Pub. No.: US 2020/0368408 A1
(43) Pub. Date: Nov. 26, 2020

(54) MANUALLY ACTIVATED NEGATIVE PRESSURE THERAPY SYSTEM WITH PRESSURE SENSORS

(71) Applicant: KCI Licensing, Inc., San Antonio, TX (US)

(72) Inventors: Richard Daniel John COULTHARD, Verwood (GB); Christopher Brian LOCKE, Bournemouth (GB)

(21) Appl. No.: 16/958,229

(22) PCT Filed: Sep. 18, 2018
(86) PCT No.: PCT/US2018/051417

§ 371 Date: Jun. 26, 2020

Related U.S. Application Data
(60) Provisional application No. 62/611,324, filed on Dec. 28, 2017.

(51) Int. Cl. A61M 1/00

(52) U.S. Cl. CPC A61M 1/0088; A61M 1/0027; A61M 1/0031; A61M 2205/073; A61M 2205/8206; A61M 2205/581; A61M 2205/583; A61M 2205/582; A61M 2205/587; A61M 2205/3344

(57) ABSTRACT

Negative-pressure therapy systems and methods can implement a feedback module within a pump which includes multiple sensors and fluid passageway(s). Another aspect of a negative-pressure therapy system and method include a feedback module including multiple pressure sensors and/or an electrical circuit within a manually-operably pump where the module is located between a compressible end cap or inlet nozzle and a charging chamber. A further negative-pressure therapy system and method provide a pump coupled to a wound dressing where the pump includes a first sensor which operably senses negative pressure from the dressing, a second sensor which operably senses if a blockage is present at the dressing or tubing therefrom, and at least a third sensor which operably senses pressure associated with a charging chamber of the pump.

### RELATED APPLICATION

This application claims the benefit, under 35 U.S.C. § 119(e), of the filing of U.S. Provisional Patent Application Ser. No. 62/611,324, entitled “MANUALLY ACTIVATED NEGATIVE PRESSURE THERAPY SYSTEM WITH PRESSURE SENSORS,” filed Dec. 28, 2017, which is incorporated herein by reference for all purposes.

### TECHNICAL FIELD

The invention set forth in the appended claims relates generally to tissue treatment systems and more particularly, but without limitation, to a manually-actuated negative-pressure therapy system with pressure sensors.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for providing negative-pressure therapy are set forth in the appended claims. Illustrative embodiments are also provided to enable a person skilled in the art to make and use the claimed subject matter.

For example, in some embodiments, negative-pressure therapy systems and methods as described herein can include a feedback module within a manually-actuated pump. Another aspect of a negative-pressure therapy system and method may include a feedback module with pressure sensors and an electrical circuit within a manually-operable pump, where the feedback module is located between a compressible end cap or inlet nozzle and a charging chamber. Some embodiments of a negative-pressure therapy system and method may provide a pump coupled to a dressing. The pump may include a first sensor that operably senses pressure from the dressing, a second sensor that can operably sense if a blockage is present at the dressing or tubing therefrom, and a third sensor which can operably sense vacuum pressure associated with a charging chamber of the pump.

Yet another aspect may employ a controller or electrical circuit, which can operably receive feedback signals from pressure sensors, compare the signals to one or more threshold values, and activate one or more indicators if the comparison results differ from the threshold valves. In some examples, a feedback module may monitor pressure within a charging chamber of the pump and provide a visual and/or audio indication of the pressure; monitor usage of the pump to determine a remaining usable period of the pump and provide a visual and/or audio indication of the remaining usable period; monitor the pressure within a charging chamber to determine whether the pressure is greater than an over-pressure threshold or is less than an under-pressure threshold and provide a visual and/or audio indication of the condition; provide an indication of remaining battery life; store data related to the operation of the pump in memory and selectively provide the data to a user or some combination of these functions.

A method of manufacturing a negative pressure therapy system, including a pump with a sensor manifold or module, is also provided.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a perspective view of an example embodiment of a negative pressure therapy system;

FIG. 2 is a section view, taken along line 2-2 in the dressing of FIG. 1, illustrating additional details that may be associated with some embodiments of the negative pressure therapy system of FIG. 1;

FIG. 3 is an exploded view of a pump, illustrating additional details that may be associated with some embodiments of the negative pressure therapy system of FIG. 1;

FIG. 4 is an exploded view of an example feedback module, illustrating additional details that may be associated with some embodiments of the pump of FIG. 3;

FIG. 5 is a front perspective view of an upper portion of the pump of FIG. 3;

FIG. 6 is an exploded view of the upper portion of FIG. 5;

FIG. 7 is a section view of the example pump of FIG. 1, taken along line 7-7, illustrating additional details that may be associated with some embodiments;

FIG. 8 is a section view of the example pump of FIG. 1, taken along line 8-8, illustrating additional details that may be associated with some embodiments of pump with an end cap uncompressed;

FIG. 9 is a section view of an upper portion of a pump with a compressed end cap, illustrating additional details that may be associated with some embodiments;

FIG. 10 is a functional block diagram of an example feedback module, illustrating additional details that may be associated with some embodiments; and

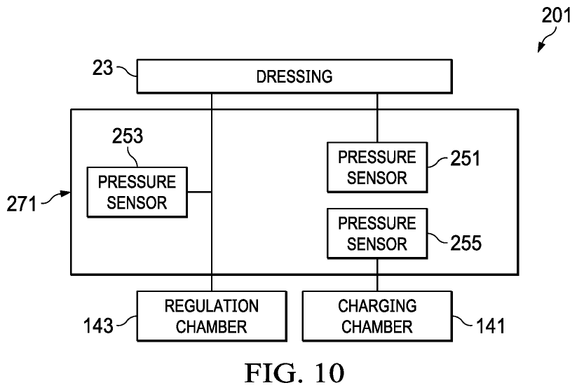

FIG. 11 is a flow diagram illustrating operations that may be associated with some embodiments of the feedback module of FIG. 4.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

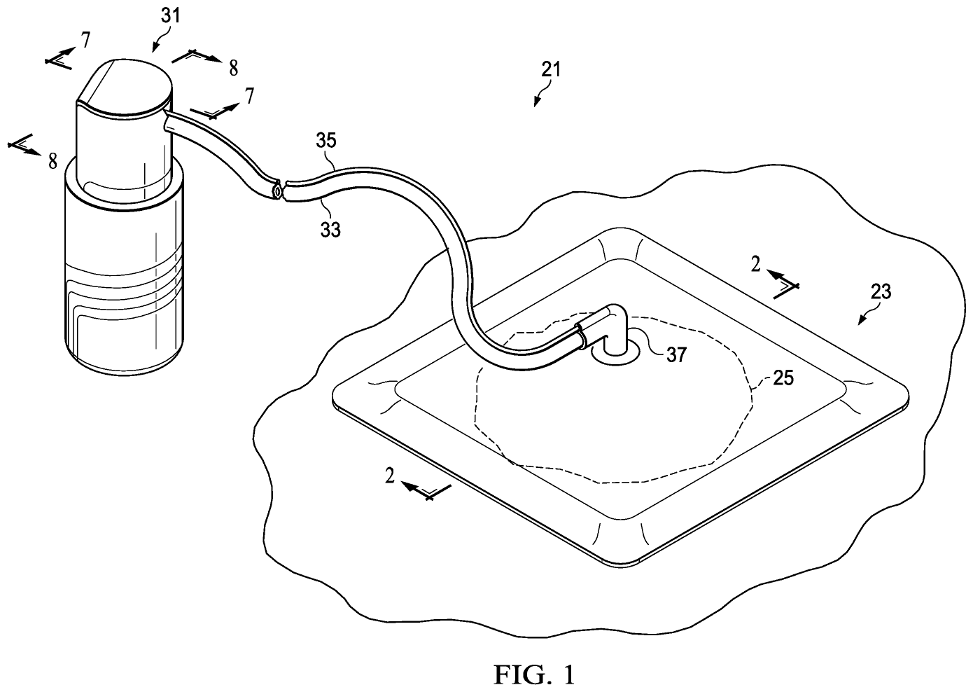

FIG. 1 is a perspective view of an example embodiment of a therapy system 21 that can provide negative-pressure therapy to a tissue site in accordance with this specification.

The term “tissue site” in this context broadly refers to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

As illustrated in the example of FIG. 1, some embodiments of the therapy system 21 may include a dressing 23 positioned at a tissue site 25. The dressing 23 may be fluidly coupled to a negative-pressure source 31. For example, a tube 33 and a tube 35 may fluidly couple the dressing 23 to the negative-pressure source 31 in some embodiments. The tube 33 and the tube 35 can fluidly communicate with the dressing 23 through a bifurcated hollow tubing adapter 37.

The negative-pressure source 31 of FIG. 1 is a manually-actuated pump. In some embodiments, the negative-pressure source 31 may include pressure regulation capabilities and may initially be charged or re-charged to a selected reduced pressure. For example, the negative-pressure source 31 may be charged by an external negative-pressure source, such as an electrically driven pump or wall-suction source, for example.

“Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

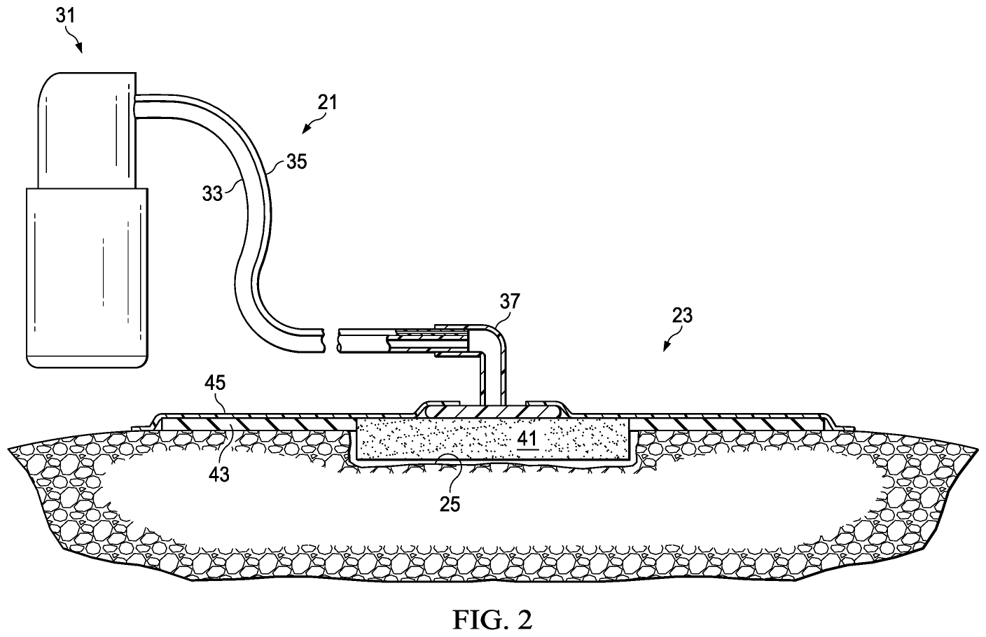

FIG. 2 is another view of the therapy system 21 with a section view of the dressing 23. In the example of FIG. 2, the dressing 23 includes a tissue interface 41 adapted to be positioned at the tissue site 25, and a sealing layer 43 adapted to seal the dressing 23 to tissue proximate the tissue site 25. A cover 45 may be positioned over the tissue interface 41 and the sealing layer 43. The cover 45 can extend beyond a perimeter of the tissue site 25 to tissue adjacent the tissue site 25.

The tissue interface 41 can be generally adapted to contact a tissue site. The tissue interface 41 may be partially or fully in contact with the tissue site. If the tissue site is a wound, for example, the tissue interface 41 may partially or completely fill the wound, or may be placed over the wound. The tissue interface 41 may take many forms, and may have many sizes, shapes, or thicknesses depending on a variety of factors, such as the type of treatment being implemented or the nature and size of a tissue site. For example, the size and shape of the tissue interface 41 may be adapted to the contours of deep and irregular shaped tissue sites. Moreover, any or all of the surfaces of the tissue interface 41 may have projections or an uneven, course, or jagged profile that can induce strains and stresses on a tissue site, which can promote granulation at the tissue site.

In some embodiments, the tissue interface 41 may be a manifold. A “manifold” in this context generally includes any substance or structure providing a plurality of pathways adapted to collect or distribute fluid across a tissue site under pressure. For example, a manifold may be adapted to receive negative pressure from a source and distribute negative pressure through multiple apertures across a tissue site, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, the pathways of a manifold may be interconnected to improve distribution or collection of fluids across a tissue site. In some illustrative embodiments, a manifold may be a porous foam material having interconnected cells or pores. For example, cellular foam, open-cell foam, reticulated foam, porous tissue collections, and other porous material such as gauze or felted mat generally include pores, edges, and/or walls adapted to form interconnected fluid channels. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

The average pore size of a foam may vary according to needs of a prescribed therapy. For example, in some embodiments, the tissue interface 41 may be a foam having pore sizes in a range of 400-600 microns. The tensile strength of the tissue interface 41 may also vary according to needs of a prescribed therapy. In one non-limiting example, the tissue interface 41 may be an open-cell, reticulated polyurethane foam such as GRANUFOAM™ dressing or V.A.C. VERAFLO™ dressing, both available from KCl of San Antonio, Tex.

The tissue interface 41 may be either hydrophobic or hydrophilic. In an example in which the tissue interface 41 may be hydrophilic, the tissue interface 41 may also wick fluid away from a tissue site, while continuing to distribute negative pressure to the tissue site. The wicking properties of the tissue interface 41 may draw fluid away from a tissue site by capillary flow or other wicking mechanisms. An example of a hydrophilic foam is a polyvinyl alcohol, open-cell foam such as V.A.C. WHITEFOAM™ dressing available from KCl of San Antonio, Tex. Other hydrophilic foams may include those made from polyether. Other foams that may exhibit hydrophilic characteristics include hydrophobic foams that have been treated or coated to provide hydrophilicity.

The tissue interface 41 may further promote granulation at a tissue site when pressure within the sealed therapeutic environment is reduced. For example, any or all of the surfaces of the tissue interface 41 may have an uneven, coarse, or jagged profile that can induce microstrains and stresses at a tissue site if negative pressure is applied through the tissue interface 41.

In some embodiments, the tissue interface 41 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 41 may further serve as a scaffold for new cell-growth, or a scaffold material may be used in conjunction with the tissue interface 41 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 45 may provide a bacterial barrier and protection from physical trauma. The cover 45 may also be constructed from a material that can reduce evaporative losses and provide a fluid seal between two components or two environments, such as between a therapeutic environment and a local external environment. The cover 45 may be, for example, an elastomeric film or membrane that can provide a seal adequate to maintain a negative pressure at a tissue site for a given negative-pressure source. The cover 45 may have a high moisture-vapor transmission rate (MVTR) in some applications. For example, the MVTR may be at least 300 g/m\hat{ }2 per twenty-four hours in some embodiments. In some example embodiments, the cover 45 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained.

An attachment device may be used to attach the cover 45 to an attachment surface, such as undamaged epidermis, a gasket, or another cover. The attachment device may take many forms. For example, an attachment device may be a medically-acceptable, pressure-sensitive adhesive that extends about a periphery, a portion, or an entire sealing member. In some embodiments, for example, some or all of the cover 116 may be coated with an adhesive, such as an acrylic adhesive, having a coating weight between 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel. In some embodiments, an adhesive disposed on the cover 45 may be used in lieu of the seal layer 43. In other embodiments, the seal layer 43 may be used in conjunction with an adhesive of the cover 45 to improve sealing of the cover at the tissue site 25. In other embodiments, the seal layer 43 may be used in lieu of adhesive disposed on the cover 45.

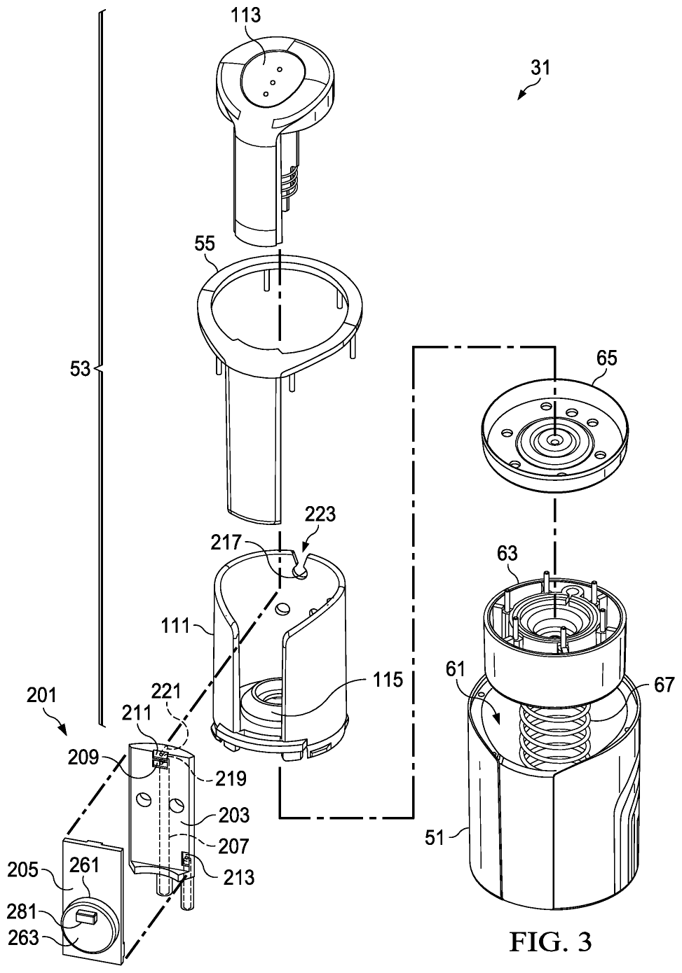

Referring to the examples of FIGS. 3-9, the negative-pressure source 31 may include an outer housing, such as an outer barrel 51, and an inner housing, such as an inner barrel 53. While the outer barrel 51 and the inner barrel 53 are illustrated as having substantially cylindrical shapes, the shapes of the barrels could be other shapes that permit operation of the device. The negative-pressure source 31 may further include a barrel ring 55, which can be positioned at an open upper end of the outer barrel 51 to circumscribe or surround the inner barrel 53. The barrel ring 55 can eliminate gaps between the outer barrel 51 and the inner barrel 53 at the open end of the outer barrel 51. The outer barrel 51 may include a hollow cavity 61 having an open upper end and defined by a substantially cylindrical interior wall surface. The cavity 61 can slidingly receive the inner barrel 53 therein.

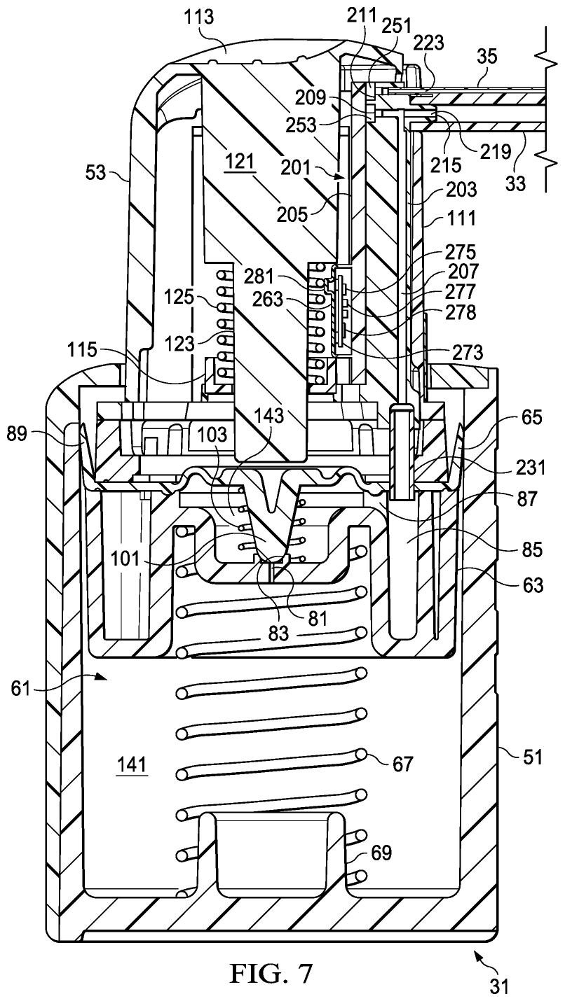

The negative-pressure source 31 may further include a piston 63 and a seal 65 in some examples. The piston 63 and the seal 65 may be slidingly received within the cavity 61 between a bottom of the inner barrel 53 and a bottom wall of the outer barrel 51, as illustrated in the example of FIG. 7. The seal 65 may be positioned between the inner barrel 53 and the piston 63.

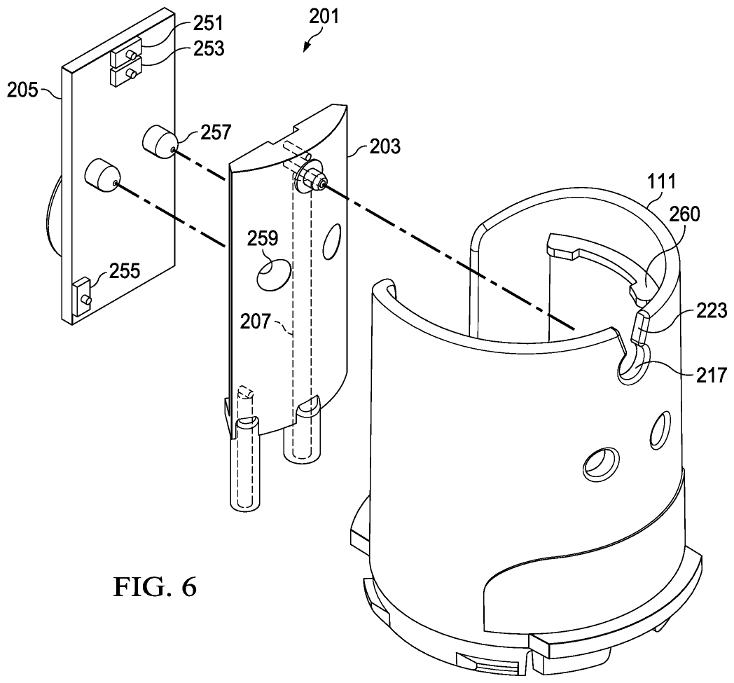

A piston spring 67 or other biasing member may be positioned within the cavity 61, and a protrusion 69, centrally projecting from the bottom wall of outer barrel 51, can receive an end of the piston spring 67. The piston 63 can receive an opposite end of the piston spring 67. The piston spring 67 can bias the piston 63, the seal 65, and an end cap 113 toward an extended and uncompressed position as illustrated in the example of FIG. 6.

In the example of FIG. 7, a regulator passage 81 is located through an inner floor of the piston 63. Furthermore, a valve seat 83 is positioned in an inner bowl of the piston 63 near the regulator passage 81 such that fluid communication through the regulator passage 81 may be selectively controlled by selective engagement of the valve seat 83 with a valve body 101. A well 85 can be positioned in an annulus of the piston 63 and a channel 87 can extend between the well 85 and the inner bowl. The channel 87 can allow fluid communication between the well 85 and the inner bowl.

In some examples, the seal 65 may include a central portion peripherally circumscribed by a circular skirt 89. An aperture 91 of the seal 65 can permit fluid communication through the seal 65 and with the well 85 of the piston 63. The skirt 89 can engage an inner surface of the outer barrel 51, as illustrated in the example of FIG. 7, to permit unidirectional fluid communication past the seal 65. More specifically, the skirt 89 can allow fluid to flow past the skirt 89 if the fluid flow is directed from the side of the seal 65 on which the piston 63 is disposed, toward the opposite side of the seal 65. Conversely, the skirt 89 can substantially prevent or deter fluid flow in the opposite direction. While the skirt 89 can effectively control fluid communication past the skirt, a valve member such as, for example, a check valve or other valve may alternately be used to control fluid flow.

The valve body 101 of FIG. 7 depends from the central portion of the seal 65 in an axial direction opposite the skirt 89. Although valve bodies of many types, shapes and sizes may be used, the valve body 101 of FIG. 7 is cone-shaped with an apex that is adapted to sealingly engage the valve seat 83 of the piston 63. While the valve body 101 is illustrated as being an integral part of the seal 65, the valve body 101 may alternately be a separate component from the seal 65. Both the seal 65 and the valve body 101 are preferably made from a flexible elastomeric material, which includes without limitation, a medical grade silicone.

A regulator spring 103 can bias the valve body 101 away from the piston 63 and the valve seat 83. One end of the regulator spring 103 can be positioned concentrically around the valve seat 83 within the inner bowl of the piston 63, while another end of the regulator spring 103 can be positioned around the valve body 101. A biasing force provided by the regulator spring 103 can urge the valve body 101 toward an open position in which fluid communication is permitted through the regulator passage 81. In some embodiments, if the spring 103 biases the valve body 101 toward the open position, only the central portion of the seal 65 moves upward due to the flexibility of the seal 65.

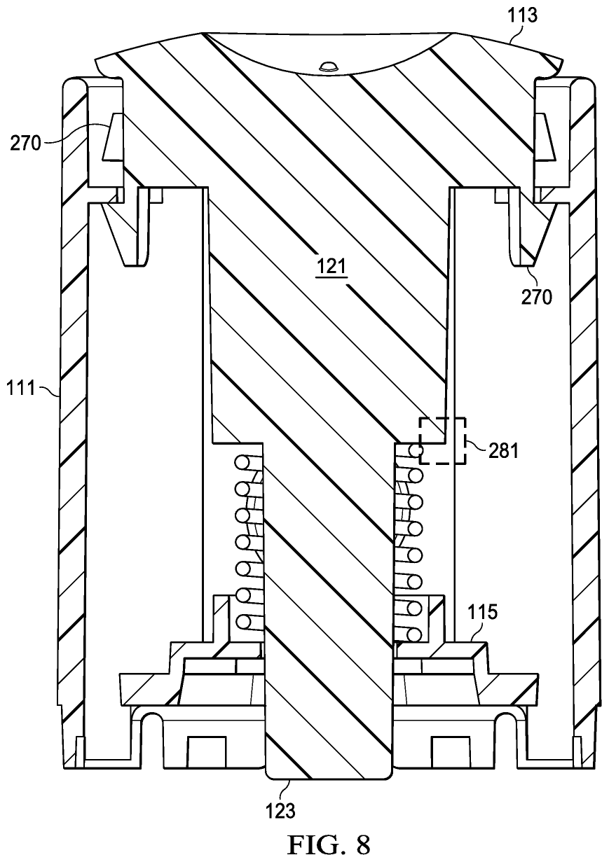

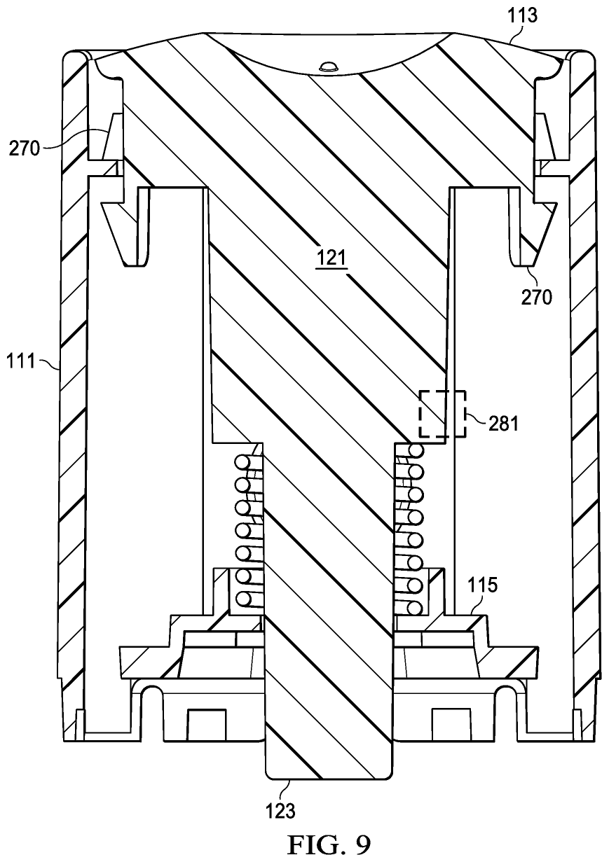

The inner barrel 53 may include a shell 111 and the end cap 113, the end cap 113 serving as a user compressible button. A floor 115 may be integrally formed with or otherwise connected to the shell 111. Barbed snap fits 270 can moveably secure the end cap 113 to an internal ledge 260 of the shell 111, and act as linear travel stops as is shown in FIGS. 8 and 9.

In some embodiments, a shaft 121 can internally and centrally extend from the end cap 113. In the example of FIG. 7, the shaft includes an engagement end 123 opposite the end cap. Furthermore, the shaft 121 may be substantially coaxial to a longitudinal axis and compression direction of the inner barrel 53 and extend through a central passage in the floor 115. One end of a compression spring 125 can bear upon the floor 115 of the shell 111 and another end of the spring 125 can bear upon a shoulder of the shaft 121. The spring 125 can bias the shaft 121 and the end cap 113 toward the uncompressed and disengaged position, in which the engagement end 123 of the shaft 121 does not bear upon the seal 65 or the valve body 101. The sliding relationship and engagement between the shell 111 and the end cap 113 can allow a user to exert a force on the end cap 113 (against the biasing force of the spring 125) to move the end cap to an engaged position. In the engaged position, the engagement end 123 of the shaft 121 can bear upon the seal 65 above the valve body 101, which can force the valve body against the valve seat 83 and prevent fluid communication through the regulator passage 81.

In some embodiments, a charging chamber 141 is defined within the cavity 61 of the outer barrel 51 between the piston 63 and the bottom wall of the outer barrel. Moreover, a regulation chamber 143 can be defined within the inner bowl of the piston 63 beneath the seal 65 in some examples. The regulator passage 81 can allow selective fluid communication between the charging chamber 141 and the regulation chamber 143 depending on the position of the valve body 101. The regulation chamber 143 can fluidly communicate with the well 85 of the piston 63 through the channel 87.

To charge or prime the pump negative-pressure source, the end cap 113 can be manually compressed into the outer barrel 51 by a user's thumb. The force exerted by the user on the end cap 113 can overcome the biasing force provided by the piston spring 67. The force being exerted on the end cap 113 by the user can also be transmitted to the seal 65 and the piston 63. The movement of the inner barrel 53, the seal 65, and the piston 63 into a compressed position decreases the volume of the charging chamber 141. As the volume of the charging chamber 141 decreases, the pressure in the charging chamber 141 can increase, but air and other gases within the charging chamber 141 can escape past the skirt 89 of the seal 65 due to the increased pressure within the charging chamber 141.

If a user releases the compressive force exerted upon the end cap 113, the biasing force exerted by the piston spring 67 on the piston 63 can move the piston 63, the seal 65, and the end cap 113 toward an extended position. As this movement occurs, the volume of the charging chamber 141 increases. Since the skirt 89 of the seal 65 allows only unidirectional flow, air and other gases cannot enter the charging chamber 141 past the skirt 89. A resulting drop in pressure (i.e., increased negative-pressure) can occur within the charging chamber 141 as the volume increases. The amount of negative pressure generated within the charging chamber 141 can be dependent on the spring constant of the piston spring 67 and the integrity of the seal 65. In some embodiments, a negative-pressure that is greater (i.e., a lower absolute pressure) than the therapy pressure to be supplied to the tissue site can be generated. For example, if the therapy pressure is 125 mm Hg of negative pressure, the charging chamber 141 can be charged to 150 mm Hg of negative pressure.

The regulation chamber 143 can be configured to regulate pressure from the charging chamber 141 to a regulated pressure, which may correspond to a desired therapy pressure that is delivered to a treatment port and the tissue site. If the negative pressure within the charging chamber 141 is greater than the negative pressure within the regulation chamber 143 and if the negative pressure in the regulation chamber 143 is less than the desired therapy pressure, the upward force on the seal 65 (exerted by the increased absolute pressure in the regulation chamber 143 and the biasing force of the regulator spring 103, both against the atmospheric pressure exerted downward on the seal 65) can move the valve body 101 into an open position and allow fluid communication between the charging chamber 141 and the regulation chamber 143. The charging chamber 141 continues to charge the regulation chamber 143 with negative pressure (i.e., the absolute pressure in the regulated chamber continues to drop) until the negative pressure in the regulated chamber, balanced against the atmospheric pressure above the seal 65, is sufficient to counteract the biasing force of the regulator spring 103 and move the valve body 101 into a closed position. Additional details of the structure and function of a suitable outer barrel, inner barrel, piston and seal can be found in the commonly owned international patent application serial number PCT/US17/18129 entitled “Manually Activated Negative Pressure Therapy System with Integrated Audible Feedback” which was filed on Feb. 16, 2017, and is incorporated by reference herein.

Reference should now be made to FIGS. 3-7 in which an example embodiment of a feedback module 201 is illustrated. The feedback module 201 includes a support board 203 and a monitoring board 205, which are both longitudinally elongated along the direction of compression. The support board 203 has a generally semicircular and arcuate exterior surface corresponding to the inside surface of the shell 111. The feedback module 201 can be secured to shell 111 by ledges 260 in some embodiments. The internally-facing surface of the support board 203 may be generally flat, as illustrated in the example of FIG. 3 and FIG. 7, and may have a bottom lip and sensor recesses, such as a first sensor recess 209, a second sensor recess 211, and a third sensor recess 213. In some embodiments, at least one longitudinally elongated, central passageway 207 may be internally located within the support board 203. In other embodiments, the passageway 207 may be defined by a semicircular depression in the support board and a flat surface of the monitoring board 205, for example.

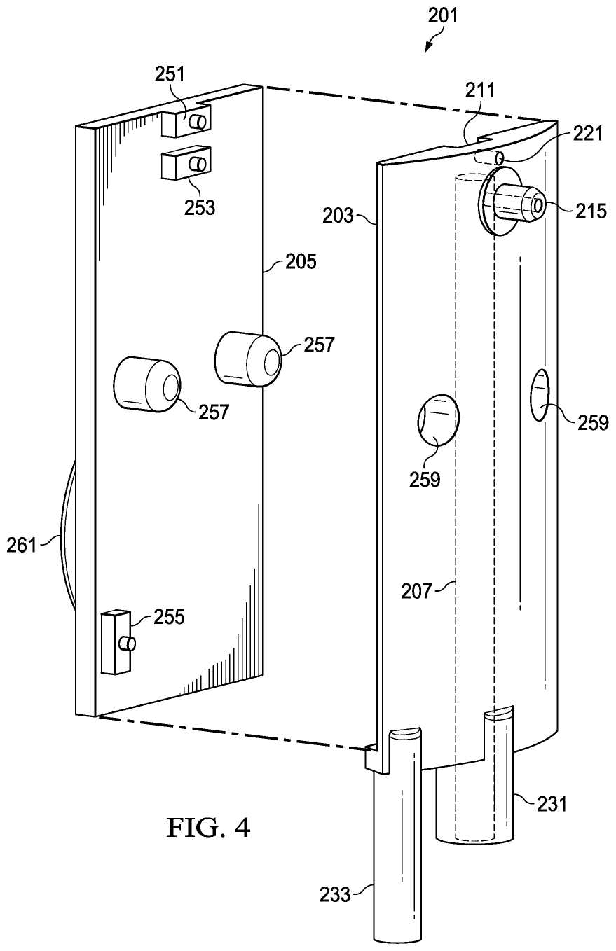

As illustrated in the example of FIG. 4, some embodiments of the feedback module 201 may include a treatment port, such as an inlet fitting 215, which may be a generally cylindrical fitting that projects outwardly from the outer surface of the support board 203. The inlet fitting 215 may extend through a corresponding hole 217 in the shell 111 in some examples. The inlet fitting 215 may have a first inlet passageway 219, which can be fluidly accessible to the first sensor recess 209, and in turn, to the central passageway 207. An elongation axis of the first inlet passageway 219 can be essentially perpendicular to that of the central passageway 207 in some embodiments. Furthermore, a feedback port may be fluidly accessible to the second sensor recess 211. For example, the feedback port may be a second inlet passageway 221, and optional connector or nozzle, may have an axis generally parallel to the first inlet passageway 219, and may be fluidly accessible to the second sensor recess 211. The second sensor recess 211 may be disposed above the first sensor recess 209 with a wall between, for example. A generally V-shaped notch 223 through the shell 111 can allow access to the second inlet passageway 221. The tube 33 can be coupled to the inlet fitting 215, and the tube 35 may be coupled to the second inlet passageway 221.

A first conduit 231 can project from a bottom edge of the support board 203, as illustrated in the example of FIG. 4. The conduit 231 may have an internal hollow bore that can fluidly couple the central passageway 207 with the well 85 of the piston 63. A second conduit 233 may also longitudinally projects from the bottom edge of the support board 203. A first end of an internal passageway through the second conduit 233 can be fluidly coupled to the third sensor recess 213, and a second end can be fluidly coupled to the charging chamber 141. The support board 203, monitoring board 205, the inlet fitting 215, the first conduit 231, and the second conduit 233 may be injection molded from a polymeric material in some examples, and may be fastened together by adhesives, snap-fit barbed fingers and grooves, sonic welding, or the like. Additional sealants, o-rings, or seals may additionally be provided at mating surfaces.

Additionally or alternatively, the first conduit 231, the second conduit 233, or both can be separately injection or extrusion molded then attached to the support board 203. In some embodiments, the first conduit 231 and the second conduit 233 may be flexible or of differing cross-sectional shapes. Moreover, at least some portion of internal passageways of the first conduit 231, the second conduit 233, or both may be defined by elongated portions of the support board 203 or the monitoring board 205. Multiple and differently oriented passageways may be disposed within some embodiments of the feedback module 201.

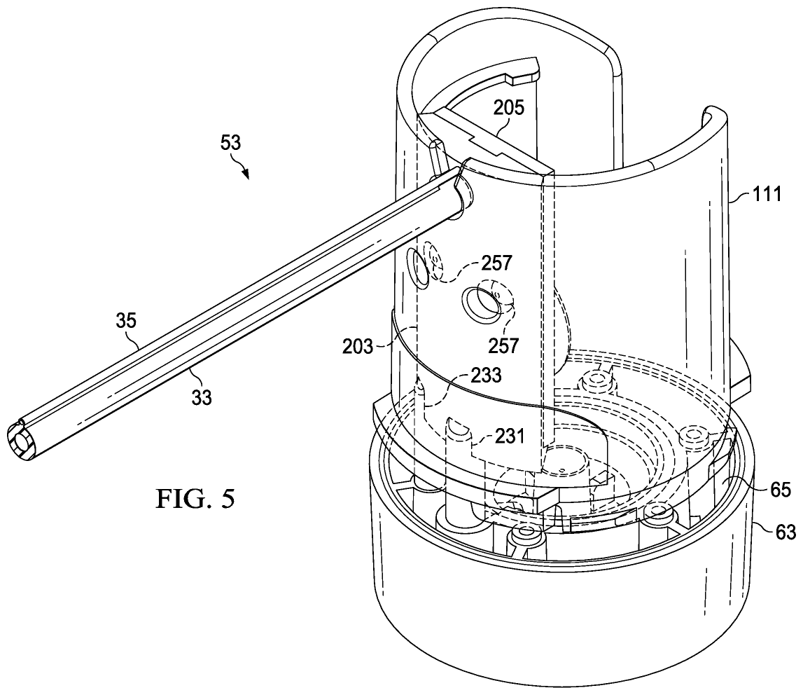

Sensors may be mounted on the monitoring board 205 in some embodiments. For example, an applied pressure sensor 251, a supply pressure sensor 253, and a charging pressure sensor 255 may be mounted on an internal face of the monitoring board 205. The supply pressure sensor 253 may be aligned with and accessible within the first sensor recess 209; the applied pressure sensor may be aligned with and accessible within the second sensor recess 211; and the charging pressure sensor 255 may be aligned with and accessible within the third sensor recess 213. At least one and preferably multiple indicators may also be mounted to the monitoring board 205 in some embodiments. For example, two indicators 257, such as light-emitting diodes (“LEDs”), may be mounted to the same face of the monitoring board 205 as the sensors. The visual indicators 257 may project through aligned apertures 259 in the support board 203 and the shell 111 so as to be visible to an operator, as illustrated in the example of FIG. 5. Additionally, or alternately, an audible indicator or a haptic indicator can be mounted and electrically connected to the monitoring board 205.

The feedback module 201 may also include an electrical circuit 271 in some embodiments. The electrical circuit 271 may include a printed circuit board (“PCB”) 273 upon which can be mounted electrical traces, a microprocessor 275, a ROM and/or RAM memory chip 277, and optionally, other electronic components. A power source, such as a battery 278, may be connected to the printed circuit board 273. Electrically conductive paths, such as a metal stamping, printed circuit, discrete wire or the like, can be mounted to the monitoring board 205 and connect the printed circuit board 273 to the applied pressure sensor 251, the supply pressure sensor 253, the charging pressure sensor 255, and the indicators 257. The printed circuit board 273 may also be disposed within a casing 261, which can be integrally molded upon the monitoring board 205 in some examples. A polymeric cover 263 can be sealingly fastened to the casing 261 such as by an adhesive, snap-fit, sonic weld or the like. The monitoring board 205 can serve as a preassembled, and self-contained single sub-module for all of the electrical items employed with the feedback module 201.

An optional electrical switch 281, as shown in the examples of FIGS. 3 and 7-9, may be mounted to the monitoring board 205, such as on the casing 261 or the cover 263. This optional switch 281 may be a proximity switch, limit switch, piezo-electric switch or the like. The switch 281 can directly or indirectly sense and detect compression of the end cap 113, the seal 65, the piston 63, or some combination of the end cap 113, the seal 65, and the piston 63 relative to the outer barrel 51 and can send a signal output to the microprocessor 275 indicative of the compression. A comparison of FIGS. 8 and 9 illustrate the shoulder of the shaft 121 physically activating the switch 281 as it is linearly compressed. Alternatively or additionally, a magnetic or conductive metal actuation member may be mounted to and moveable with the shaft 121, which can activate the switch 281, if a proximity switch, as it moves past, or these parts can be reversed. In some examples, receipt of an output signal from the switch 281 can cause the microprocessor 275 to electrically wake up or energize the electrical circuit 271 from a battery conserving sleep mode. Such a sleep mode may occur during transportation or storage of the system, for example.

The electrical circuit 271 can optionally and further include a countdown timer on the printed circuit board 273 that can begin when the switch 281 activates the electrical circuitry upon initial charging of the negative-pressure source 31. After a predetermined period of time has elapsed, the microprocessor 275 can activate the indicators 257 to alert the user that the useful life of the negative-pressure source 31 has then been exhausted. In an optional variation, the microprocessor 275 can disable the negative-pressure source 31 when the countdown timer period has expired or ended. Such an automatic disablement can be achieved by use of a bi-stable valve that is normally closed, and is opened by the microprocessor 275 after timer expiration to connect the therapy delivery or vacuum channel to atmospheric pressure. Such a bi-stable valve requires power during a transition between positions so that it would not prematurely drain the battery during normal pump use. Once triggered, the bi-stable valve can remain open even if the battery is subsequently depleted. In another alternate variation, the expiration valve can be incorporated into a thin section or film located adjacent to either the therapy delivery or vacuum passageways. A heating element can optionally be mounted to a surface of this thin section or film such that upon timer expiration, an electrical current is applied to the heating element which would then soften the thermoplastic thin section or film to allow it to be frangibly broken or ruptured when a pneumatic pressure differential is applied.

Another alternate deactivation timed structure can allow atmospheric venting from either the therapy delivery or vacuum channels. For example, a vent can be normally sealed by a transparent film held in place with an adhesive, whose properties can be altered by external stimuli such as exposure to light or a predetermined frequency. The film and stimuli generator can be internal to a pump and adjacent to the control board where a light-emitting diode is located. The light emitting diode may be operated by the microprocessor 275 to emit light at a frequency that can weaken and open the vent through rupture of the film.

To minimize battery usage and conserve power, the microprocessor 275 may not monitor the pressure on a continual basis, but rather sample the pressure periodically. For example, the microprocessor 275 may sample the pressure once every one minute or five minutes. In some embodiments the electrical circuit 271 may operate in the sleep mode (e.g., a mode where the components are powered off or supplied a lower power level) for a predetermined period and transition to an awake mode to sample the pressure and respond accordingly, and then transition back to the sleep mode for the predetermined period.

In some alternate embodiments, the electrical circuit 271 may include an RFID antenna to provide communications between the microprocessor 275 and a user and/or device external to the negative-pressure source 31. For example, an RFID antenna may communicate data stored in the memory to the user in response to queries. The data may include, but is not limited to, a time and date that the pump was first activated, a total period the pump has been at a desired reduced pressure, a total period the pump has not been charged to the desired reduced pressure (and/or has been not charged at all), a number of times the pump has been charged, an estimated remaining life of the pump, an estimated remaining power in the battery, an actual pressure versus desired threshold difference, or the like.

In some embodiments, some or all of the pressure sensors, indicator lights and other electrical components may be located between the compressible endcap 113 and the charging chamber 141, and more preferably at or between the second inlet passageway 221 and the valve seat 83 of the piston 63. Furthermore, in some embodiments, the electronic and circuits and electrical components may be mounted to a single monitoring board 205, which can advantageously allow for easier and lower cost preassembly and testing in a modularized manner. Alternately, one or more of the sensors, switch, indicators or other electrical components can be mounted to other items of the negative-pressure source, or the feedback module 201 may include the electrical circuitry but not the air passageways, or vice versa.

The negative-pressure source 31 may be manually actuated in some embodiments, which can significantly reduce power consumption. Some embodiments may consume only a few milliwatts of battery power, and the battery weight and size can be reduced to increase portability. For example, a 3V coin-cell battery having a weight of less than 3 grams may be suitable for some embodiments.

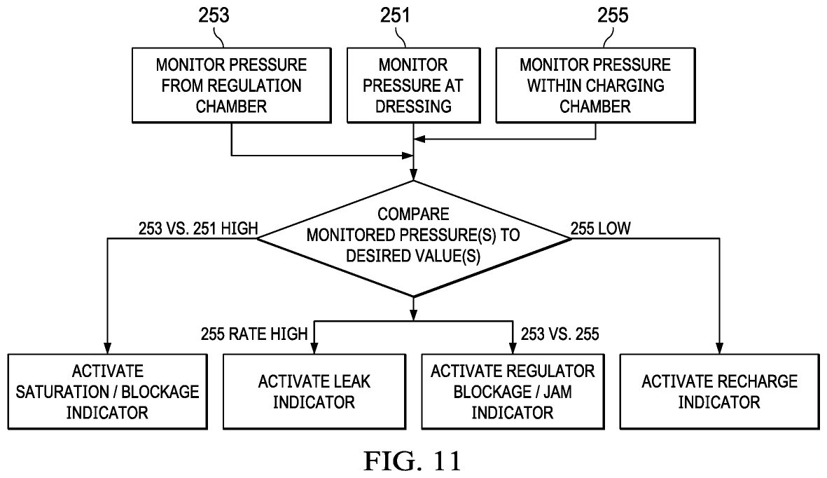

FIG. 11 is a flow diagram illustrating operations that may be associated with some embodiments of the feedback module of FIG. 4. The programmed microprocessor controller 275 can operably monitor the pressure delivered from the negative-pressure source 31, the pressure received at the dressing 23, and the pressure contained within the charging chamber 141, as sensed by the pressure sensors 251, 253 and 255. For example, the controller 275 may be configured to receive feedback signals from one or more of the applied pressure sensor 251, the supply pressure sensor 253, and the charging pressure sensor 255, and to generate an alert signal based on at least one of the feedback signals. For example, as illustrated in FIG. 11, the controller 275 can compare these pressure readings to determine and calculate if there is sufficient pressure in the therapy system 21 and if so, if it is actually being delivered to the dressing 23. Thereafter, the microprocessor 275 can cause one or more of the indicators 257 to alert an operator if a problem is detected, such as by flashing or changing colors of the indicators 275. More specifically, the microprocessor 275 and the sensor 255 can monitor pressure in the charging chamber 141. The microprocessor 275 and the sensor 251 can also monitor pressure on the tissue side of the dressing 23 as a proxy for pressure at the wound site. Furthermore, the microprocessor 275 and the sensor 253 can monitor pressure at the output of the regulation chamber 143. In normal operation, the pressure at the dressing 23 and at an output of the regulation chamber 143 should be equal and in the designed range.

The microprocessor 275 can receive a sensor output signal and determine if the pressure in the charging chamber 141 is below the desired therapy pressure value or threshold. If the calculated comparison differs from the desired threshold value or range then the microprocessor 275 can cause the indicators 257 to inform an operator that the piston may be fully extended and may be compressed to recharge the negative-pressure source 31. The microprocessor 275 can also determine if the pressure difference between the sensor 251 the sensor 253 is greater than a predetermined limit or threshold value. For example, the microprocessor 275 can determine if the dressing 23 is saturated or the tubing is blocked based on the comparison, in which event the microprocessor 275 can cause the indicators 257 to inform an operator that the dressing should be changed.

The microprocessor 275 can also determine if the rate of pressure change in the charging chamber 141 is greater than a predetermined limit or threshold value. For example, the rate of pressure change in the charging chamber 141 may exceed a threshold if there is a leak in excess of a desired limit or threshold range, in which event the microprocessor 275 can cause the indicators 257 to provide a leak signal. Additionally, the microprocessor 275 can determine if there is a pressure difference between the charging chamber 141 and the regulation chamber 143 based on a calculated comparison of sensed values. If this occurs then the microprocessor 275 can determine that the regulator has a problem, such as blocked or stuck, in which event the microprocessor 275 can cause the indicators to provide a regulator error signal.

A method of manufacturing an embodiment of the negative-pressure source 31 may include creating (such as by molding) a first housing including a moveable end cap; creating (such as by molding) a second housing from a polymeric material including the bottom wall, side wall and charging chamber; assembling a piston within the charging chamber; assembling the first housing to the second housing to allow relative movement between the end cap and the second housing; creating passageway(s) with a feedback module; assembling the multiple sections of the module together; and mounting the module inside the pump with all of the sensors being located between at least one external air inlet and the charging chamber.

The systems, apparatuses, and methods described herein may provide significant advantages. For example, the negative-pressure 31 may be a manually-operated pump, which can significantly reduce power requirements and cost compared to electrically-powered pumps. Manually-operated pumps can also increase mobility, which may be particularly beneficial for patients with low-acuity wounds. A manually-actuated pump also works well for wound treatment where there is no hospital infrastructure accessible to the patient or where there is a limited supply of medical equipment. The feedback module 201 may enhance these advantages by providing alerts and information to an operator, without significantly increasing power requirements. For example, some embodiments of the feedback module 201 may determine when a dressing is full and alert an operator, which may be particularly beneficial for dressings used under compression garments. The negative-pressure source 31 may also significantly reduce noise that can be produced by electrically-powered pumps, providing relatively unobtrusive therapy.

While shown in a few illustrative embodiments, a person having ordinary skill in the art will recognize that the systems, apparatuses, and methods described herein are susceptible to various changes and modifications. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, additional and/or alternately functioning sensors may be mounted to the feedback module. Furthermore, additional and/or alternate passageways may be present within the feedback module.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

1. A system for providing negative-pressure therapy, the system comprising:
a dressing;
a pump comprising:
at least one housing comprising a moveable end cap;
at least one inlet passageway fluidly coupled to the dressing; and
pressure sensors located internal to the at least one housing, at least one of the pressure sensors being located adjacent to the at least one inlet passageway and adjacent to the moveable end cap.

2. The system of claim 1, further comprising:
a piston; and
the at least one housing comprising a first barrel and a second barrel which are coupled together in a stacked relationship, the second barrel including a charging chamber therein between the piston and a bottom wall of the second barrel.

3. The system of claim 2, wherein all of the pressure sensors are located between an exterior surface of the end cap and the charging chamber.

4. The system of claim 2, further comprising a feedback module having a passageway between the at least one inlet passageway and the charging chamber, the pressure sensors being coupled to the feedback module.

5. The system of claim 1, further comprising:
a feedback module disposed within the pump and comprising at least one passageway;
wherein there are at least three of the pressure sensors which are all mounted within the feedback module.

6. The system of claim 1, further comprising:
a feedback module located within the at least one housing;
a first of the at least one inlet passageway being a nozzle outwardly projecting from the feedback module and being accessible to a first of the pressure sensors;
a second of the at least one inlet passageway being part of the feedback module and being accessible to a second of the pressure sensors; and
at least one tube coupling the first and the second of the at least one inlet passageway to the dressing.

7. The system of claim 1, further comprising:
a feedback module located within the at least one housing, the feedback module having at least one passageway internally extending from the at least one inlet passageway to a negative-pressure chamber within the at least one housing;
an electrical circuit mounted to the feedback module, the electrical circuit comprising a microprocessor and a battery; and
a visual indicator mounted to the feedback module and visible from outside the pump, being powered by the battery and activated by the microprocessor in response to a condition sensed by at least one of the sensors.

8. The system of claim 1, wherein:
a first of the pressure sensors is configured to operably sense negative pressure from the dressing;
a second of the pressure sensors is configured to operably sense if a blockage is present at the dressing;
a third of the pressure sensors is configured to operably sense pressure from a charging chamber within the pump; and
an indicator light being located between at least two of the pressure sensors.

9. The system of claim 1, further comprising:
a feedback module located within the pump upon which at least one of the pressure sensors is mounted;
a printed circuit board, including a microprocessor, mounted to the feedback module;
a switch mounted on the feedback module configured to be actuated to send a signal to the microprocessor that wakes up an electrical circuit from a sleep mode and/or activates a timer; and
a longitudinally elongated shaft internally extending from and moving with the end cap, the end cap being manually compressible to charge the pump and actuate the switch with the shaft.

10. A manually compressible negative-pressure pump comprising:
an end cap having an external surface;
a housing including a bottom wall and a charging chamber;
a piston moveable within the charging chamber in response to the end cap being compressed toward the housing;
a feedback module located within the pump, the feedback module comprising:
at least one internal passageway;
multiple sensors located between the external surface of the end cap and the charging chamber;
an electrical circuit comprising a microprocessor and an electrical power source located between the external surface of the end cap and the charging chamber;
an indicator operably activated by the microprocessor in response to a condition sensed by at least one of the sensors.

11. The pump of claim 10, wherein there are at least three internal passageways and the sensors include at least three pressure sensors aligned with at least three of the internal passageways of the feedback module.

12. The pump of claim 11, wherein:
a first of the sensors is configured to operably sense negative air pressure from a wound dressing;
a second of the sensors is configured to operably sense if a blockage is present at the wound dressing; and
a third of the sensors is configured to operably sense pressure in the charging chamber.

13. The pump of claim 10, further comprising:
a tube-receiving nozzle projecting from the feedback module adjacent the end cap, the nozzle projecting through a hole in an inner barrel extending from the housing.

14. The pump of claim 13, wherein:
the housing and the inner barrel have substantially cylindrical external shapes coaxially aligned with each other; and
the indicator is a light which is visible through a hole in the inner barrel.

15. The pump of claim 10, wherein:
the feedback module is longitudinally elongated in a direction of compression of the pump;
the feedback module comprises at least two elongated mating sections with at least one of the internal passageways being internal to a first of the sections located closest to an inlet nozzle; and
all of the sensors, electrical circuit and indicator are mounted to a second of the sections.

16. The pump of claim 10, further comprising:
a printed circuit board, including a programmable controller and a battery, being mounted on an inwardly facing surface of the feedback module;
an inlet passageway laterally extending through an outwardly facing surface of the feedback module; and
the at least one internal passageway including a longitudinally elongated passageway extending internally within the feedback module between the inwardly and outwardly facing surfaces, one end of the longitudinally extending passageways being in fluid communication with the inlet passageway, and an opposite end of the longitudinally extending passageway being adjacent the piston.

17. A negative-pressure therapy system comprising:
a dressing including a tissue interface and a sealing layer;
a manually-actuated pump comprising:
a manually moveable cap;
a charging chamber;
a piston moveable within the charging chamber in response to movement of the cap;
a feedback module located internally within the pump, the feedback module comprising:
multiple passageways;
multiple sensors;
an electrical circuit including a controller and a battery;
a visual indicator operably activated by the controller in response to a condition sensed by at least one of the sensors;
the feedback module being longitudinally elongated in a direction of movement of the piston;
the feedback module including at least two elongated sections with at least one of the passageways being located within a first of the sections;
an elongated tube connecting the tissue interface to the at least one of the passageways of the first module section; and
all of the sensors, electrical circuit and indicator are mounted to a second of the module sections.

18. The system of claim 17, wherein:
a first of the sensors is configured to operably sense negative pressure from the dressing;
a second of the sensors is configured to operably sense if a blockage is present at the dressing; and
a third of the sensors is configured to operably sense pressure from the charging chamber.

19. The system of claim 17, further comprising a second tube coupling the dressing to the feedback module adjacent an end of the feedback module opposite that to which the controller is mounted.

20. The system of claim 17, wherein the sensors are pressure sensors and the visual indicator includes multiple light emitting diodes visible through spaced apart holes in an outer housing of the pump.

21. The system of claim 17, wherein all of the sensors are located between an exterior surface of the cap and the charging chamber.

22. The system of claim 17, wherein the controller is configured to:
receive a signal from a first of the sensors to monitor pressure from a regulation chamber of the pump;
receive a signal from a second of the sensors to monitor pressure at the dressing;
receive a signal from a third of the sensors to monitor pressure within the air charging chamber;
compare values associated with the monitored pressure signals to desired values;
activate the indicator, which acts as a saturation or blockage indicator, when an associated comparison differs beyond a threshold;
activate the indicator, which acts as a leak indicator, when an associated comparison differs beyond a threshold;
activate the indicator, which acts as a regulator blockage indicator, when an associated comparison differs beyond a threshold; and
activate the indicator, which acts as a recharge indicator, when an associated comparison differs beyond a threshold.

23.-43. (canceled)
